(19) United States
(12) Patent Application Publication
(10) Pub. No.: US 2024/0366965 A1
(43) Pub. Date: Nov. 7, 2024

(54) MULTI-FOCUS HISTOTRIPSY METHOD AND SYSTEM BASED ON PULSED ULTRASOUND PHASED ARRAY WITH HUNDRED ELEMENTS

(71) Applicant: XI'AN JIAOTONG UNIVERSITY, Xi'an (CN)

(72) Inventors: Mingzhu LU, Xi'an (CN); Xuan LIU, Xi'an (CN); Rongzheng YANG, Xi'an (CN); Ruixin LI, Xi'an (CN); Jianyun MAO, Xi'an (CN); Yi ZHANG, Xi'an (CN); Quan ZHANG, Xi'an (CN); Tingting QI, Xi'an (CN); Mingxi WAN, Xi'an (CN)

(21) Appl. No.: 18/674,806

(22) Filed: May 24, 2024

Related U.S. Application Data
(63) Continuation of application No. PCT/CN2021/138964, filed on Dec. 17, 2021.

(30) Foreign Application Priority Data
May 28, 2021 (CN) 202110588064.8

(51) Int. Cl. A61N 7/00

(52) U.S. Cl. CPC A61N 7/00; A61N 2007/0039; A61N 2007/0073; A61N 2007/0078

(57) ABSTRACT

A multi-focus histotripsy method based on a pulsed ultrasound phased array with hundred elements is provided. Excitation amplitude and phase of individual elements are regulated to control a phased array to produce multiple focus modes. A two-stage histotripsy is performed based on these focus modes. In a first stage, a first pulsed ultrasound is applied to an experimental sample in a target area to induce generation of cavitation microbubbles and boiling bubbles to preliminarily homogenize the experimental sample to form a loose product; and in a second stage, the experimental sample is mechanically disrupted and homogenized using a second pulsed ultrasound. The first pulsed ultrasound and the second pulsed ultrasound each are a hundred micro-second pulse or a milli-second pulse. Duty cycles of the first pulsed ultrasound and the second pulsed ultrasound are 3-10% and less than 2%, respectively. A multi-focus histotripsy system is further provided.

### CROSS-REFERENCE TO RELATED APPLICATIONS

This application is a continuation of International Patent Application No. PCT/CN2021/138964, filed on Dec. 17, 2021, which claims the benefit of priority from Chinese Patent Application No. 202110588064.8, filed on May 28, 2021. The content of the aforementioned application, including any intervening amendments thereto, is incorporated herein by reference in its entirety.

### TECHNICAL FIELD

This application relates to focused ultrasound, and more specifically to a multi-focus histotripsy method and system based on a pulsed ultrasound phased array with hundred-elements.

### BACKGROUND

Histotripsy is a non-invasive focused ultrasound therapy. It uses ultrasound waves, cavitation microbubbles or boiling bubbles to mechanically disrupt (liquefy) the target tissue, which is conducive to the postoperative absorption, and can overcome the heat sink. Therefore, it has become a research hot spot in the field of ultrasound therapy. In the histotripsy, the pulsed ultrasound cavitation or boiling bubbles are used to mechanically liquefy the target tissue into subcellular debris or homogenize it into emulsions that can be easily absorbed by other tissues, so as to achieve the precise treatment without damaging the surrounding normal tissues. In addition to the ablation of solid tumors, the histotripsy has also been extensively used in the treatment of deep venous thrombosis (DVT), intracranial hematoma, benign prostatic hyperplasia, and congenital heart disease. At present, the histotripsy mainly includes cavitation cloud histotripsy (CH) and boiling histotripsy (BH).

The CH is proposed by Zhen Xu et al. from the University of Michigan, which uses micro-second ultrasound pulses to generate a large number of cavitation microbubbles in the focal zone. The microbubbles will be gathered to form a cavitation cloud, which further experience rapid expansion, contraction and violent collapse to produce strong mechanical strain and stress to mechanically disrupt the surrounding cells into subcellular debris, thus realizing the target tissue homogenization. U.S. Pat. No. 6,309,355B1 filed by Cain et al., titled “Method and assembly for performing ultrasound surgery using cavitation”, discloses a treating method and apparatus using ultrasound-induced cavitation. The memory effect of the cavitation cloud will lead to cavitation activity in the pre-focal region and the peripheral region of the focal zone, thereby damaging the surrounding normal tissues and significantly reducing the histotripsy efficiency. U.S. Patent Publication No. 2013/0090579A1 filed by Cain et al., titled “Pulsed cavitational therapeutic ultrasound with dithering”, discloses that the cavitation memory can be eliminated by removing the cavitation nuclei through increasing the inter-pulse interval.

The BH is proposed by Khokhlova et al. from the University of Washington, which uses milli-second pulses to rapidly heat the focal zone and generate boiling bubbles. The shock wave interacts with the boiling bubbles to create an atomization effect at the boiling bubble-tissue interface, forming a micro-fountain to be ejected toward the inside of the cavity to fractionate the target tissue into subcellular debris. U.S. Pat. No. 8,876,740B2 filed by Michael S. Canney et al., titled “Methods and systems for non-invasive treatment of tissue using high intensity focused ultrasound therapy”, describes a method and apparatus for generating boiling bubbles in a target tissue using pulsed ultrasound. Further, US Patent Publication No. 20170072227A1 filed by Vera Khokhlova et al., titled “Boiling histotripsy methods and systems for uniform volumetric ablation of an object by high intensity focused ultrasound waves with shocks”, discloses a method for sequentially focusing on different parts of a tissue to achieve a larger-area and uniform histotripsy.

The excitation sequence proposed in the above studies of CH and BH is simple repetition of pulsed ultrasound of different durations. However, the pulsed ultrasound sequences used for histotripsy can be greatly optimized to fully take the kinetic processes involving the generation, sustaining, motion, and dissipation of cavitation microbubbles and boiling bubbles into account, so as to enhance the histotripsy efficacy.

In addition, the therapeutic transducers used in these studies are generally single-element focusing transducers, which can only irradiate a very small focal area at a time, and requires mechanical scan when treating a large tumor area, leading to a low efficiency of histotripsy. To increase the focal volume and shorten the treatment time, using a split array transducer is an improved strategy.

Although the use of a split array expands the volume of the focal zone, the split array generally has a small number of array elements, and therefore can only form a fixed focus area, and the focus cannot be shifted and scanned. In contrast to the split array transducer, the phased array transducer can generate multiple foci at the same time to increase the treatment area, and can also generate a flexible and versatile variety of focus patterns depending on the shape and size of the lesion, including scanning of the foci, which requires changes in the amplitude and phase of the drive signals of individual array elements.

In summary, the histotripsy has more advantages and broader clinical application prospects than the traditional thermal ablation therapy. However, the existing histotripsy methods still have the following defects. The excitation sequence of histotripsy is a simple repetition of pulsed ultrasound with a certain duty cycle, which does not make full use of the characteristics of cavitation microbubbles and boiling bubbles induced in the focal zone. Therefore, the histotripsy efficiency needs to be further improved. In addition, the histotripsy generally adopts a single-element therapeutic transducer, which can only produce a small focal point, such that multiple irradiations are required to fractionate large tumors, resulting in longer treatment time.

### SUMMARY

Concerning the above-mentioned issues, the present disclosure proposes a multi-focus histotripsy method and system based on a pulsed ultrasound phased array with hundred elements. The method provided herein adopts a more efficient two-stage histotripsy pulse sequence, and further combines it with a phased-array transducer to temporally and spatially improve the histotripsy efficiency.

Technical solutions of the present disclosure are as follows.

In a first aspect, this application provides a multi-focus histotripsy method based on a pulsed ultrasound phased array with hundred elements, comprising:
regulating the excitation amplitude and the phase of each of the hundred elements according to demands to control the phased-array transducer to produce a plurality of multi-focus modes; and
performing a two-stage histotripsy based on the multi-focus modes through the following steps:
   in a first stage, applying a first pulsed ultrasound to an experimental sample in a target area to induce generation of cavitation microbubbles and boiling bubbles to preliminarily homogenize the experimental sample to form a loose product; and
   in a second stage, applying a second pulsed ultrasound to mechanically disrupt and homogenize the loose product;
   wherein the first pulsed ultrasound is configured to employ a first hundred micro-second length pulse or a first milli-second length pulse, and the second pulsed ultrasound is configured to employ a second hundred micro-second length pulse or a second milli-second length pulse; and a duty cycle of the first pulsed ultrasound is 3-10%; and a duty cycle of the second pulsed ultrasound is less than 2%.

In some embodiments, when the first pulsed ultrasound employs a first hundred micro-second length pulse, and the second pulsed ultrasound employs a second hundred micro-second length pulse, a pulse repetition frequency (PRF) of the first hundred micro-second length pulse is 40-300 Hz; an individual pulse duration (PD) is 300-900 μs; and the number of repetitions S1 for each group of pulses is 100-900. A PRF of the second hundred micro-second length pulse is 40-300 Hz; an individual PD is 300-900 μs; a 400-900 ms off-time is set after every 15-30 groups of pulses are applied; and the number of repetitions S2 of combined pulses is 15-35.

In some embodiments, when the first pulsed ultrasound employs a first milli-second length pulse, and the second pulsed ultrasound employs a second milli-second length pulse, a PRF of the first milli-second length pulse is 8-20 Hz; an individual PD is 2-10 ms; and the number of repetitions S1 for each group of pulses is 15-90. A PRF of the second milli-second length pulse is 8-20 Hz; an individual PD is 2-10 ms; a 1-5 s off-time is set after every 8-30 groups of pulses are applied; and the number of repetitions S2 of combined pulses is 4-10.

In some embodiments, the transducer for histotripsy is a phased-array transducer, which is provided with a circular hole at the center for placing an ultrasound monitoring probe.

In some embodiments, the phased array is controlled to generate different focus modes through steps of: calculating an acoustic field of the phased array; and controlling the focus mode of the phased array using an optimization algorithm;
wherein the acoustic field of the spherical phased array is calculated through the following formula:

$$P = \frac{j ρ c k}{2 π} {\sum }_{n = 1}^{N} {u}_{n} \frac{{F}_{n} Δ A}{{R}_{n}} {e}^{- ( α + j k ) {R}_{n}} sin c \frac{k {x}_{s n} Δ w}{2 R} sin c \frac{k {y}_{s n} Δ h}{2 R} ; \qquad ( 1 )$$

wherein ΔW represents an element width; Δh represents an element height; ΔA represents an element area; the origin of an xyz coordinate system is at the apex of a spherical cap; a beam direction is set as z-axis; j=\sqrt{−1}; ρ represents a density of a medium; c represents an acoustic velocity in the medium; k represents the wave number, and k=ω/c; N represents the number of elements; and uₙ represents the velocity of the surface mass point of the n^{th} element as the element driving signal;

$${R}_{n} = \sqrt{{( x - {x}_{n} )}^{2} + {( y - {y}_{n} )}^{2} + {( z - {z}_{n} )}^{2}} ; \qquad ( 2 )$$

$${F}_{n} = \frac{{R}_{S R}}{\sqrt{{R}_{SR}^{2} - ( {x}_{n}^{2} + {y}_{n}^{2} )}} ; \qquad ( 3 )$$

$$R = \sqrt{{( x - {x}_{n} )}^{2} + {( y - {y}_{n} )}^{2} + {z}^{2}} ; \qquad ( 4 )$$

$${R}_{zn}^{2} = {R}_{SR}^{2} - {( {x}_{n} + {y}_{n} )}^{2} ; \qquad ( 5 )$$

$${x}_{s n} = x + \frac{z - {R}_{S R}}{{R}_{z n}} {x}_{n} ; \qquad ( 6 )$$

$$and {y}_{s n} = y + \frac{z - {R}_{S R}}{{R}_{z n}} {y}_{n} ; \qquad ( 7 )$$

the focus mode of the phased array is controlled using the optimization algorithm through steps of:
writing the formula (1) into a matrix form:

$${P}_{M} = {H}_{M} {u}_{N} ; \qquad ( 8 )$$

wherein M represents the number of focus points, and N represents the number of elements;
finding an inverse of the matrix P_{M} as a driving signal u_{N}:

$${u}_{N} = {{H}_{M}^{τ} ( {H}_{M} {H}_{M}^{τ} )}^{- 1} {P}_{M} ; \qquad ( 9 )$$

and
combining the acoustic field calculation method and the optimization algorithm to obtain multiple focus modes of the phased array.

In some embodiments, an optimal drive signal for individual elements of the phased-array transducer under the plurality of multi-focus modes is calculated using a genetic algorithm to produce acoustic field distributions of a single-focus mode, a dual-focus mode and a four-focus mode through the following steps:
encoding solutions into chromosomes to form an initial population;
subjecting the initial population to evolution from generation to generation through a series of genetic operations including selection-replication, crossover and mutation to gradually approach an optimal solution;
in each generation, (1) evaluating the current chromosome by calculating the fitness function, with the sound intensity gain as the fitness function Fit:

$$Fit \{ {θ}_{i} \} = \frac{{P}_{M}^{τ} {P}_{M}}{{u}_{N}^{τ} {u}_{N}} = \frac{{P}_{M}^{τ} {P}_{M}}{{{P}_{M}^{τ} ( {H}_{M} {H}_{M}^{τ} )}^{- 1} {P}_{M}} ; \qquad ( 10 )$$

(2) replicating some chromosomes with the largest fitness values; wherein during the selection-replication process, each individual reproduces its offspring according to its fitness; during the crossover, two chromosomes are randomly selected for crossover with a random crossover point, and the number of offspring produced by the crossover depends on the crossover probability; and during the mutation, the individual to be mutated is randomly selected to randomly change the number of bits in each generation according to the mutation probability; and
(3) repeating the above steps to continuously generate new generations until the termination criterion is satisfied to obtain [θ(1), θ(2), . . . , θ(M)] corresponding to the optimal focusing control under different focus modes; and forming a P_{M} vector based on [θ(1), θ(2), . . . , θ(M)] and a preset P_{M} amplitude, and calculating the element driving signal—under different focus modes according to formula (9).

In some embodiments, before the two-stage histotripsy based on the plurality of multi-focus modes, a position of the experimental sample is adjusted to the focal zone of the phased array through steps of:
inducing a thermal damage in the experimental sample using a continuous wave mode; intersecting two laser beams at a position suffering the thermal damage, with an intersection point serving as an approximate focal point of the phased-array transducer; and moving the experimental sample to the approximate focal point using a three-dimensional (3D) positioning subsystem.

In a second aspect, this application provides a multi-focus histotripsy system based on a pulsed-ultrasound phased array with hundred elements, comprising:
a transducer-wave driver subsystem;
wherein the transducer-wave driver subsystem comprises a phased-array transducer with hundred elements and a driver; the driver is connected to the phased-array transducer through an impedance matching network, wherein each of the hundred elements is connected to an independent driving channel.

In some embodiments, the system further comprises:
a high-speed camera subsystem;
a data acquisition subsystem;
a passive cavitation detection (PCD) acoustic signal detection subsystem; and
the 3D positioning subsystem;
wherein the PCD acoustic signal detection subsystem comprises a PCD probe, a wideband receiver, a data acquisition card and a first computer; and the PCD probe, the wideband receiver, the data acquisition card and the first computer are electrically connected in sequence; and
the 3D positioning subsystem comprises a 3D driving device and a second computer; the second computer is a control computer; the 3D driving device is electrically connected to the second computer; and the experimental sample is adapted to be set on the 3D driving device, and to be placed at the approximate focal point of the phased-array transducer.

Compared with the prior art, the present application has at least the following beneficial effects.

(1) Compared with the existing histotripsy excitation sequences, the excitation sequence proposed herein is a two-stage pulsed ultrasound sequence, where at the first stage, a pulsed ultrasound with a higher duty cycle is applied to the target tissue to make the target tissue structurally loose and porous, and the connection become fragile, and at the second stage, a pulsed ultrasound with a lower duty cycle is applied to the target tissue to completely homogenize the target tissue. The pulsed ultrasound sequence designed herein can effectively utilize the activities of cavitation microbubbles and boiling bubbles to reduce the ultrasound excitation time required to cause damage and thus improve the histotripsy efficiency.

(2) Further, the present disclosure proposes two different lengths of ultrasound pulses: a hundred-microsecond pulse, which mainly utilizes the cavitation cloud formed by the shock wave for histotripsy; and a millisecond pulse, which mainly performs histotripsy through the activity of the boiling bubbles.

(3) Compared with the histotripsy realized based on a single-array transducer, the therapeutic transducer proposed in the present disclosure is a phased-array transducer, which can realize multiple focusing modes by electronic control without mechanical scanning to move the position of the focal zone. The multiple focus mode can effectively treat large-volume areas, shorten the treatment time, and spatially improve the efficiency of histotripsy.

In summary, the method provided herein can temporally and spatially increase the histotripsy efficiency effectively,

### BRIEF DESCRIPTION OF THE DRAWINGS

The present application will be described in detail with reference to the accompanying drawings and specific embodiments.

FIG. 1 is a schematic diagram of a hundred-microsecond ultrasonic pulse sequence according to an embodiment of the present disclosure, where at a first stage, a pulse repetition frequency (PRF) is 40-300 Hz, an individual pulse duration (PD) is 300-900 μs, and the number of repetitions S1 for each group of pulses is 100-900; and at a second stage, the PRF is 40-300 Hz, an individual PD is 300-900 μs, an interval after every 15-30 groups of pulses have been applied is 400-900 ms, and the number of repetitions S2 of the combined pulse is 15-35;

FIG. 2 is a schematic diagram of a milli-second ultrasonic pulse sequence according to an embodiment of the present disclosure, where at a first stage, a PRF is 8-20 Hz, an individual PD is 2-10 ms, and the number of repetitions S1 of each group of pulses is 15-90; and at a second stage, the PRF is 8-20 Hz, an individual PD is 2-10 ms, a 1-5 s off-time is set after every 8-30 groups of pulses have been applied is, and the number of repetitions S2 of combined pulse is 4-10;

FIG. 3 is a front view of a phased-array transducer according to an embodiment of the present disclosure, where 1, array element; and 2, central circular hole; as an example, the number of array elements of the phased array transducer in the figure is 256, and the array elements are periodically arranged; and it should be noted that the method proposed herein is also applicable to phased array transducers with different numbers of array elements (e.g., 512) and different array element arrangements (e.g., spiral arrangement, etc.);

FIG. 4 is a flowchart of a genetic algorithm used herein to control a phased-array multi-focus modes according to an embodiment of the present disclosure;

FIGS. 5a-b show an acoustic field performance of a phased-array single-focus mode obtained using the genetic algorithm according to an embodiment of the present disclosure, where 5a: ultrasound intensity distribution in a single-focal focus plane; and 5b: ultrasound intensity contour map in a single-focal xy plane;

FIGS. 6a-b show an acoustic field performance of a phased-array dual-focus mode obtained using the genetic algorithm according to an embodiment of the present disclosure, where 6a: ultrasound intensity distribution in a dual-focus plane; and 6b: ultrasound intensity contour map in a dual-focus xy plane;

FIGS. 7a-b show an acoustic field performance of a phased-array four-focus mode obtained using the genetic algorithm according to an embodiment of the present disclosure, where 7a: ultrasound intensity distribution in a four-focus plane; and 7b: ultrasound intensity contour map in a four-focus xy plane;

FIGS. 8a-b show an acoustic field performance of a phased-array off-axial four-focus mode obtained using the genetic algorithm according to an embodiment of the present disclosure, where 8a: ultrasound intensity distribution in an off-axial four-focus plane; and 8b: ultrasound intensity contour map in an off-axial four-focus xy plane;

FIG. 9 is a block diagram of an experimental system according to an embodiment of the present disclosure, where 1, Verasonics control software platform equipped with a channel waveform control panel and an optimization software for acquiring a drive signal of the hundred elements; 2, Verasonics hundred-channel control system, configured to execute the phase and amplitude of individual elements and ultrasonic pulse waveform set by the control panel, and with its output eventually driving the phased array HIFU transducer; 3, Verasonics high-intensity focused ultrasound (HIFU) power supply; 4, impedance matching network; 5, phased array HIFU transducer; 6, phantom; 7, water tank; 8, three-dimensional (3D) positioner control system; 9, passive cavitation detection (PCD) probe; 10, Gage acquisition card; 11, high-speed camera; and 12, computer;

FIG. 10 shows high-speed photography results during a damage formation process in a bovine-serum-protein acrylamide phantom during a two-stage histotripsy using hundred-microsecond pulsed ultrasound according to an embodiment of the present disclosure, where (a)-(d): high-speed camera results under the single-focus mode; (e)-(h): high-speed camera results under the dual-focus mode; and (i)-(l): high-speed camera results under the four-focus mode;

FIG. 11 shows high-speed photography results during a damage formation process in a bovine-serum-protein acrylamide phantom during a two-stage histotripsy using milli-second pulsed ultrasound according to an embodiment of the present disclosure, where (a)-(d): high-speed camera results in the single-focus mode; (e)-(h): high-speed camera results in the dual-focus mode; and (i)-(l): high-speed camera results in the four-focus mode;

FIGS. 12a-d show changes in mean square energy values of PCD signals with histotripsy time under phased-array single-focus mode, the dual-focus mode and the four-focus mode according to an embodiment of the present disclosure, where the mean square value of broadband signals after comb-like filtering reflects energy of transient cavitation in a focal region;

FIGS. 13a-c show histotripsy results of in vitro porcine kidney after two-stage histotripsy with a hundred microsecond pulse according to an embodiment of the present disclosure, where a-c are anatomical graphs of the in vitro porcine kidney under the single-focus mode, the dual-focus mode, and the four-focus mode, respectively; and

FIGS. 14a-c show histotripsy results of in vitro porcine kidney after two-stage histotripsy with a milli-second pulse according to an embodiment of the present disclosure, where a-c are anatomical graphs of the in vitro porcine kidney under the single-focus mode, the dual-focus mode, and the four-focus mode, respectively.

### DETAILED DESCRIPTION OF EMBODIMENTS

To enable one of ordinary skill in the art to better understand the technical solutions in the present disclosure, the technical solutions in the embodiments of the present disclosure will be clearly and completely described below with reference to the accompanying drawings. Obviously, the described embodiments are only some embodiments of the present disclosure. Based on these embodiments, all other embodiments obtained by one of ordinary skill in the art without making creative effort should fall within the scope of the present disclosure.

It should be noted that when an element is said to be “arranged on” another element, it may be directly arranged on the other element or there may be an element between the two elements. When an element is said to be “connected to” another element, it may be directly connected to the other element or there may be an element between the two elements. The terms “vertical,” “horizontal,” “left,” “right,” and similar expressions used herein are merely illustrative, and are not intended to limit the implementation of the present application.

Unless otherwise defined, all technical and scientific terms used herein have the same meaning as commonly understood by one of ordinary in the art. Terms used herein are used only for the purpose of describing specific embodiments and are not intended to limit the present disclosure. The term “and/or” as used herein includes any and all combinations of one or more of the relevant listed items.

In a first aspect, this application provides a multi-focus histotripsy method based on a pulsed ultrasound phased array with hundred elements, which mainly includes the following steps.

(1) An optimized two-stage histotripsy pulse sequence is proposed, in which a higher duty cycle pulse is used in the first stage and a lower duty cycle pulse is used in the second stage.

(2) Two driving waveforms for the two-stage histotripsy are proposed, i.e., a hundred-microsecond pulsed ultrasound and a millisecond pulsed ultrasound.

(3) A therapeutic transducer used herein for the two-stage histotripsy is a phased-array transducer.

The method specifically includes the following steps.

The excitation amplitude and the phase of each of the hundred elements are regulated according to demands to control the phased-array transducer to produce the multi-focus modes.

A two-stage histotripsy is performed based on the multi-focus modes.

In a first stage, a first pulsed ultrasound is acted on an experimental sample in a target area to induce cavitation microbubbles and boiling bubbles to preliminarily homogenize the experimental sample to form a loose product.

In a second stage, the experimental sample is mechanically disintegrated and thoroughly homogenized using a second pulsed ultrasound.

Both the first pulsed ultrasound and the second pulsed ultrasound are a hundred micro-second pulse or a milli-second length pulse. A duty cycle of the first pulsed ultrasound is 3-10%, and a duty cycle of the second pulsed ultrasound is less than 2%.

The present disclosure takes full advantage of the fact that the phased array can simultaneously generate multiple focus patterns that can be electronically scanned, combining with the hundred micro-second pulse or the milli-second length pulse to carry out the two-stage histotripsy, which improves the histotripsy efficiency in terms of space and time.

The present disclosure is described in detail below with reference to the accompanying drawings and embodiments.

The present disclosure proposes an optimized histotripsy pulse sequence to improve the histotripsy efficacy. Unlike the simple repetitive pulse sequence described above, the histotripsy process provided in the present disclosure is divided into two stages. The first stage is mainly used to change the structure of the target tissue and its mechanical properties, and initially homogenize the tissue. The second stage is mainly used to achieve further mechanical disintegration and complete homogenization on the basis of the initial homogenization of the tissue.

The difference between the two stages of the pulse sequence is in duty cycle, where the duty cycle of the pulsed ultrasound in the first stage is higher, and the duty cycle of the pulsed ultrasound in the second stage is lower. In the first stage, the duty cycle (DC) of the pulse sequence is designed to be 3-10%, mainly utilizing the higher efficiency of the mechanical effect of the pulsed ultrasound with high DC and avoiding the occurrence of thermal diffusion. In the second stage, the duty cycle of the pulse sequence is designed to be less than 2% by inserting off-time to let bubble dissolution and to render the disintegrate a clear and smooth boundary. In addition, in the second stage, the damage mode of “excitation” and “stop” is utilized. The setting of off-time is mainly used to passively eliminate the possible “cavitation memory” and to prevent non-uniform damage caused by overheating of local tissues in the focal zone.

Based on the principle of histotripsy through cavitation clouds, one way of the two-stage histotripsy provided in the present disclosure is to use a hundred micro-second length pulsed ultrasound. In the first stage, a pulse repetition frequency (PRF) of the pulsed ultrasound is 40-300 Hz; an individual pulse duration (PD) is 300-900 μs; and the number of repetitions S1 for each group of pulses is 100-900. In the second stage, a PRF of the pulsed ultrasound is 40-300 Hz; an individual PD is 300-900 μs; a 400-900 ms off-time is set after every 15-30 groups of pulses have been applied; and the number of repetitions S2 of combined pulses is 15-35.

Based on the principle of histotripsy through boiling bubbles, another way of the two-stage histotripsy provided in the present disclosure is to use a milli-second pulsed ultrasound. Compared with the hundred-microsecond pulse, the milli-second pulse has lower repetition frequency and longer pulse duration, which is more conducive to produce heat rapidly and form more and larger boiling bubbles during the damage formation process. In the first stage, a PRF of the pulsed ultrasound is 8-20 Hz; an individual PD is 2-10 ms; and the number of repetitions S1 for each group of pulses is 15-90. In the second stage, the PRF of the second pulsed ultrasound is 8-20 Hz; an individual PD is 2-10 ms; a 1-5 s off-time is set after every 8-30 groups of pulses have been applied is; and the number of repetitions S2 of combined pulse is 4-10.

Different from the single-array transducer commonly used in the histotripsy process, the present disclosure proposes a phased-array transducer with hundreds of array elements for histotripsy. The phased-array transducer has the following advantages. The amplitude and phase of each array element is independently controllable, and multiple focus modes of acoustic field distributions can be generated by regulating the excitation amplitude and phase of each array element, forming the focal zone with desired shape, size and position, thereby realizing the targeted and fit-to-shape histotripsy. Multi-focus mode can effectively increase the area of one sonication and shorten the time of histotripsy. Moreover, the phased-array transducer can electrically steer the focus position. The multiple elements are conducive to producing finer and more variable multi-focus modes with better conformability.

The present disclosure combines the above two-stage histotripsy pulse sequence with a phased-array transducer, which can make full use of the activities of cavitation microbubbles and boiling bubbles and take advantage of the flexibility and variability of the multi-focus patterns of phased-array transducer, so as to improve the histotripsy efficiency in dimensions of time and space.

In a second aspect, this application provides an experimental system for histotripsy, which includes a therapy transducer-wave driver subsystem, a high-speed photography subsystem, a data acquisition subsystem, a passive cavitation detection (PCD) acoustic signal detection subsystem, and a three-dimensional (3D) positioning subsystem. The therapy transducer-wave driver subsystem includes a phased-array transducer with hundreds of elements and a Verasonics system. The high-speed photography subsystem mainly includes a high-speed camera. The PCD acoustic signal detection subsystem comprises a PCD probe, a wideband receiver, a Gage high-speed data acquisition card and a computer. The 3D positioning subsystem comprises a 3D driving device and a control computer.

Further, a Matlab software is used to program and control relevant parameters in the Verasonics system to achieve control of the phased-array focus mode and driving waveform. The high-speed camera monitors the cavitation activity in the focus area. The PCD probe receives passive cavitation signals generated during the cavitation activity. The 3D positioning device is used to precisely move the bovine-serum-albumin (BSA) phantom or in vitro tissue to a desired position.

A multi-focus histotripsy method based on the phased-array pulsed ultrasound with hundred elements provided herein includes the following steps.

(1) An excitation amplitude and a phase of each of the hundred elements are regulated according to demands to control the phased-array transducer to produce a plurality of multi-focus modes.

(2) A position of the BSA phantom or in vitro tissue is adjusted to a focal zone of the phased array transducer.

(3) A first-stage histotripsy is performed, where a pulsed ultrasound with higher duty cycle is used to generate high-efficiency mechanical effects in the focal zone to change the local mechanical properties and structure of the target tissue.

(4) A second-stage histotripsy is performed, where a pulsed ultrasound with a lower duty cycle is used to radiate the focal zone to further mechanically disintegrate and homogenize the tissue in the damaged area.

Further, in step (1), an optimal drive signal for each of the hundred elements of the phased-array transducer under the multi-focus modes is calculated using an optimization algorithm (e.g., genetic algorithm or particle swarm optimization algorithm) to produce acoustic field distributions of a single-focus mode, a dual-focus mode and a four-focus mode.

Further, in step (2), a small thermal damage is induced on the BSA simulated phantom or isolated tissue using a continuous wave mode. Two laser beams are intersected at a position suffering the thermal damage, with an intersection point serving as an approximate focal point of the phased-array transducer. The BSA phantom or in vitro tissue is moved to the approximate focal point using the 3D positioning system.

Further, in step (3), in the first-stage histotripsy, the duty cycle (DC) of the pulse focused ultrasound is designed to be 3-10% to simultaneously produce the higher-efficiency mechanical effects, resulting in partial homogenization of the target tissue. This stage of histotripsy changes the structural and mechanical properties of the target tissue, i.e., the tissue target becomes loose and porous and the intercellular connections become fragile, which is in preparation for the second stage of complete homogenized histotripsy.

Further, in step (4), in the second-stage histotripsy, the duty cycle (DC) of the pulsed focused ultrasound is designed to be less than 2%. The pulse sequence with a low duty cycle generates a strong inertial cavitation activity, whose mechanical effect can completely disintegrate the target tissue to form a homogenized damage. The pulse sequence has a stopping time after a certain number of repetitions in order to eliminate the possible “cavitation memory”, which makes the subsequent cavitation activity more energetic.

Further, in steps (3) and (4), the ultrasonic pulse waveforms for the two-stage histotripsy have two choices: a hundred-microsecond pulse, which mainly utilizes the cavitation cloud formed by backscattering of the shock wave under the effect of the pulse to carry out the histotripsy; and a milli-second length pulse, which mainly carries out the histotripsy by enhancing the activity of boiling bubbles.

Based on the current research and application, to further improve the histotripsy efficiency, the present disclosure discloses an experimental system for the multi-focus histotripsy method based on the pulsed ultrasound phased array with hundred elements.

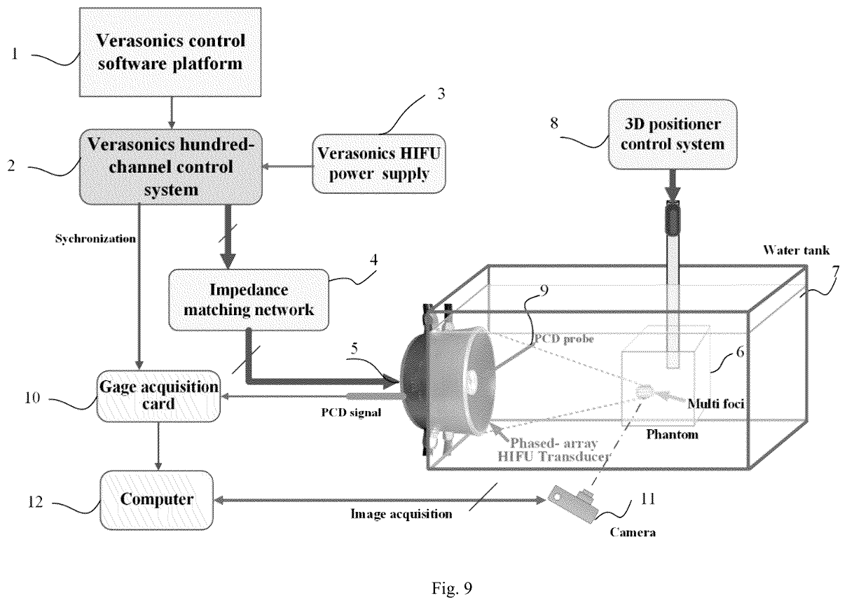

Referring to FIG. 9, the experimental system for achieving the multi-focus histotripsy method based on a pulsed ultrasound phased array with hundred elements includes a HIFU emitting unit and a monitoring unit. The HIFU emitting unit includes a Verasonics control software platform 1, a Verasonics hundred-channel control system 2, a Verasonics HIFU power supply 3, an impedance matching network 4 and a phased-array HIFU transducer 5. The Verasonics control software platform 1 includes a channel waveform control panel software and an optimization software for acquiring a drive signal of the hundred elements. The Verasonics hundred-channel control system 2 is configured to execute the phase and amplitude of individual elements and ultrasonic pulse waveform set by the control panel, and the output of the Verasonics hundred-channel control system 2 is applied to the phased-array HIFU transducer 5 through the impedance matching network 4 to drive the emission of focused ultrasound to perform histotripsy. The monitoring unit includes a phantom 6, a water tank 7, a three-dimensional (3D) positioner control system 8, a passive cavitation detection (PCD) probe 9, a Gage acquisition card 10, a high-speed camera 11 and a computer 12. The channel waveform control panel software and the optimization software in the Verasonics control software platform 1 are both programmed by MATLAB, where the channel waveform control panel software can obtain a drive control signal u_{N} (vector, where individual components are phase angle and amplitude of the voltage applied to individual elements) of the elements corresponding to multiple focuses, and can also set the ultrasonic pulse waveform. The channel waveform control panel software is operated to control the Verasonics hundred-channel control system 2 to drive the emitting elements. The phased-array element drive control signal u_{N} (vector) is inversely obtained using the genetic optimization algorithm described below. Position and amplitude of each of multiple focuses for histotripsy are designed, where the position and inter-focus distance can be arbitrarily designed. The phased-array element drive control signal u_{N} is inversely obtained by using the genetic algorithm in combination with the spherical acoustic field calculation formula, where individual components are phase angle and amplitude of the voltage applied to individual elements. The phased-array element drive control signal u_{N} is then read and executed by the channel waveform control panel software.

In the experiment, various waveforms and different focus modes of the phased array can be controlled by setting the relevant parameters in the Verasonics hundred-channel control system 2.

The PCD probe 9, the Gage acquisition card 10 and the computer 12 are electrically connected in sequence.

The 3D positioner control system 8 includes a 3D driving device and a control computer. The 3D driving device is electrically connected to the control computer. The experimental sample is set on the 3D driving device, and is placed at a focal point of the phased-array HIFU transducer 5.

The high-speed camera 11 is mainly used to reveal the physical mechanism of histotripsy, through which the kinetic process of cavitation cloud and boiling bubbles and the evolution of damage in the transparent BSA phantoms can be observed and recorded in real time.

When collecting PCD signals, the PCD probe 9 is placed at the center hole of the HIFU transducer 5, and is co-axial with the HIFU transducer 5, which can effectively receive acoustic signals during cavitation and the activity of boiling bubbles in the focal zone.

The 3D positioner control system 8 consists of a 3D driving device and a control computer, which can precisely move the transparent BSA simulated phantom or isolated tissue to the position of the focal zone of the transducer.

The multi-focus histotripsy method based on the pulsed ultrasound phased array with hundred elements is performed as follows.

(S1) An acoustic field of the phased array is calculated.

As shown in FIG. 3, the phased-array transducer may be a spherical phased array, and the acoustic field of the spherical phased array is calculated through the following formula:

$$P = \frac{j ρ c k}{2 π} {\sum }_{n = 1}^{N} {u}_{n} \frac{{F}_{n} Δ A}{{R}_{n}} {e}^{- ( α + j k ) {R}_{n}} sin c \frac{k {x}_{s n} Δ w}{2 R} sin c \frac{k {y}_{s n} Δ h}{2 R} ; \qquad ( 1 )$$

where ΔW represents an element width; Δh represents an element height; ΔA represents an element area; the origin of an xyz coordinate system is at the apex of a spherical cap; a beam direction is set as z-axis; j=\sqrt{−1}; ρ represents a density of a medium; c represents an acoustic velocity in the medium; k represents the wave number, and k=ω/c; N represents the number of elements; and uₙ represents the velocity of the surface mass point of the n^{th} element as the element driving signal:

$${R}_{n} = \sqrt{{( x - {x}_{n} )}^{2} + {( y - {y}_{n} )}^{2} + {( z - {z}_{n} )}^{2}} ; \qquad ( 2 )$$

$${F}_{n} = \frac{{R}_{S R}}{\sqrt{{R}_{SR}^{2} - ( {x}_{n}^{2} + {y}_{n}^{2} )}} ; \qquad ( 3 )$$

$$R = \sqrt{{( x - {x}_{n} )}^{2} + {( y - {y}_{n} )}^{2} + {z}^{2}} ; \qquad ( 4 )$$

$${R}_{zn}^{2} = {R}_{SR}^{2} - {( {x}_{n} + {y}_{n} )}^{2} ; \qquad ( 5 )$$

$${x}_{s n} = x + \frac{z - {R}_{S R}}{{R}_{z n}} {x}_{n} ; \qquad ( 6 )$$

$$and {y}_{s n} = y + \frac{z - {R}_{S R}}{{R}_{z n}} {y}_{n} . \qquad ( 7 )$$

(S2) The focus mode of the phased-array transducer is controlled using an optimization algorithm.

The formula (1) is redrafted in matrix form:

$${P}_{M} = {H}_{M} {u}_{N} ; \qquad ( 8 )$$

where M represents the number of focus points, and N represents the number of array elements.

An inverse of the matrix is found as a driving signal u_{N}:

$${u}_{N} = {{H}_{M}^{τ} ( {H}_{M} {H}_{M}^{τ} )}^{- 1} {P}_{M} . \qquad ( 9 )$$

In an embodiment, a genetic algorithm can be used to control the focus mode of the phased array. The genetic algorithm implementation requires the definition of two elements, i.e., chromosome and fitness function.

The chromosomes, i.e., the individuals, represent possible solutions to the target problem. The most common representation of a chromosome is a binary string, where parts of the string represent the encoded variables or parameters of the solution. Since the solution to the problem is a set of phases [θ(1), θ(2), . . . , θ(M)] of P_{M}, the chromosome in the present disclosure is defined as an 8-bit binary encoding of the phases [θ(1), θ(2), . . . , θ(M)].

The fitness function is used to evaluate the quality of the current chromosome. The sound intensity gain is used as the fitness function Fit in the present disclosure:

$$Fit \{ {θ}_{i} \} = \frac{{P}_{M}^{τ} {P}_{M}}{{u}_{N}^{τ} {u}_{N}} = \frac{{P}_{M}^{τ} {P}_{M}}{{{P}_{M}^{τ} ( {H}_{M} {H}_{M}^{τ} )}^{- 1} {P}_{M}} ; \qquad ( 10 )$$

where the maximum value of the fitness function corresponding to θ=[θ(1), θ(2), . . . , θ(M)] is the optimal solution.

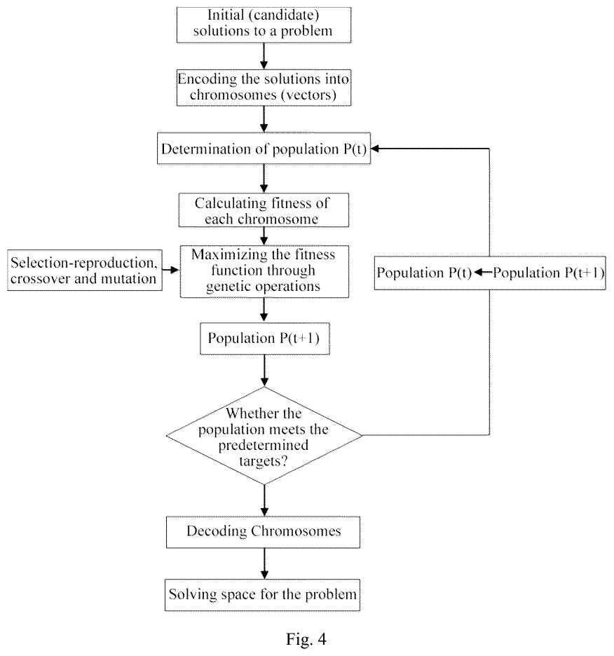

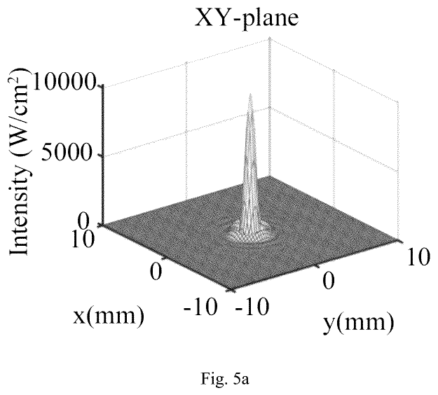

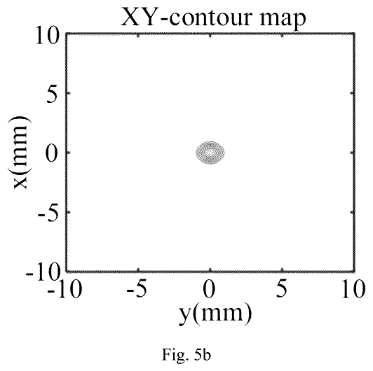

The process of the standard genetic algorithm is shown in FIG. 4. Possible solutions are encoded into chromosomes to form an initial population. The initial population is subjected to evolution from generation to generation through a series of genetic operations including selection-reproduction, crossover and mutation, to gradually approach an optimal solution.

In each generation, the current chromosome is evaluated by calculating the fitness function. Some chromosomes with the largest fitness values are replicated. During the selection-replication, individual reproduces its offspring in proportion to its fitness. During the crossover, two chromosomes are randomly selected to crossover with a random crossover point, and the number of offspring produced by the crossover depends on the crossover probability. During the mutation, the individual to be mutated is randomly selected to randomly change the number of bits in each generation with the mutation probability. The above steps are repeated to keep generating new generations until the termination criterion is satisfied to obtain [θ(1), θ(2), . . . , θ(M)] corresponding to the optimal focusing control under different focusing modes. A P_{M} vector is formed by [θ(1), θ(2), . . . , θ(M)] and a set P_{M} amplitude, and the driving signal u_{N} can be calculated by formula (9).

By combining the above acoustic field calculation methods and optimization algorithms for phased arrays, it is possible to design multiple focus modes for phased arrays.

FIGS. 5a-b show the acoustic field performance of the phased array single-focus mode in the present disclosure. The medium acoustic parameters are as follows: density p=1000 kg/m³, sound velocity c=1500 m/s, and attenuation coefficient α=0.05 Np/cm/MHz. It can be seen that the formed single focal point acoustic performance is excellent with very small side lobes and virtually no gating lobes.

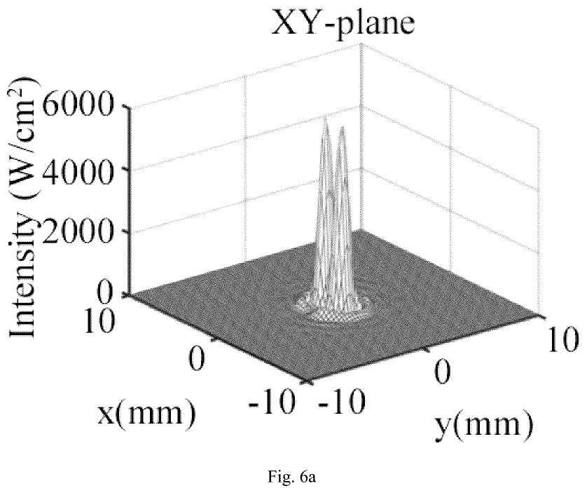

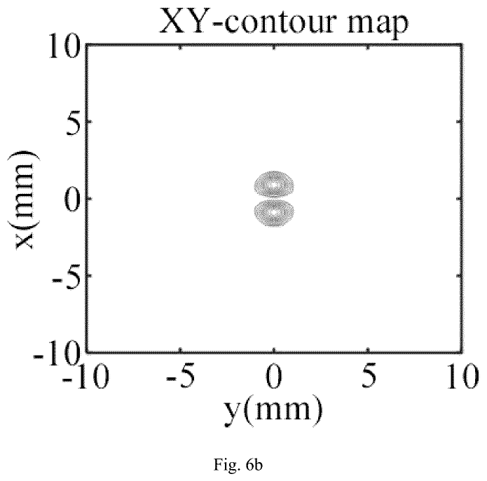

FIGS. 6a-b and 7a-b show the acoustic field performance of the phased-array multi-focus mode in the present disclosure. It can be seen that in both the dual-focus mode and the quadruple-focus mode, the individual foci are clearly distinguishable, with very small side lobes and almost no grating lobes. The multi-focus mode is the main advantage of the phased array, which can destroy larger tumor areas and thus shorten the histotripsy time.

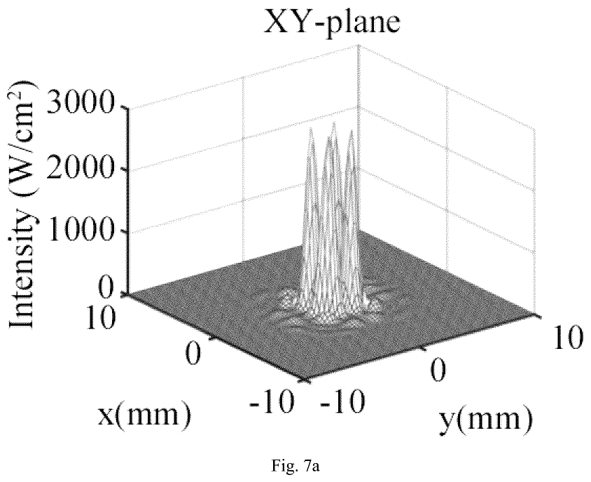

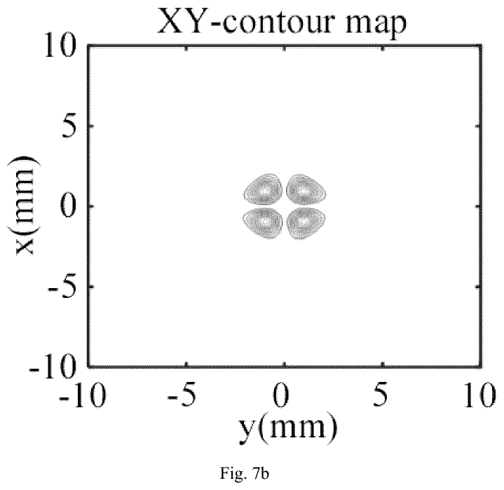

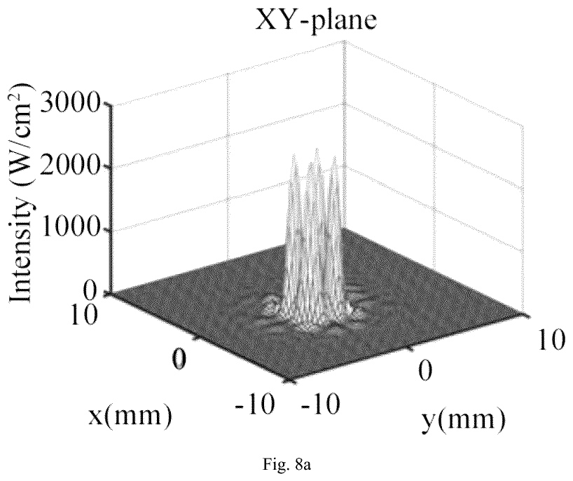

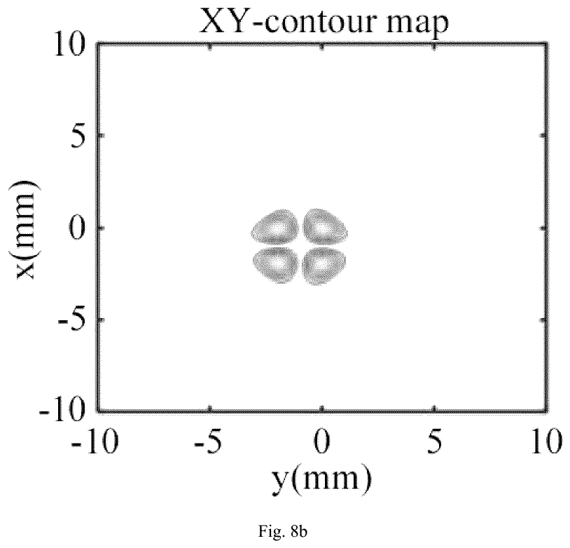

FIGS. 8a-b show the sound field performance of the phased-array off-axial-four-focus mode of the present disclosure. The position of the focal zone in FIGS. 8a-b are shifted compared to that in FIGS. 7a-b, but the four focus points can be distinguished from each other. This indicates that deflection of the focused acoustic beam and shifting of the focal point positions can be achieved by controlling the drive signal of the phased array without moving the transducer.

(S3) Two-stage pulsed ultrasound histotripsy is performed.

The driving system of the phased-array transducer is a Verasonics system, and a pulse sequence control panel is written using Matlab software. In the experiments, a two-stage pulsed ultrasound histotripsy method is used, aiming to change the local mechanical properties and structure of the target region before further destruction. The first-stage histotripsy mainly utilizes the pulsed ultrasound with higher duty cycle with high-efficiency mechanical effects to produce cavitation effects and boiling bubbles in the target area, thereby reducing the mechanical strength of the target tissue and achieving partial homogenization. The second-stage histotripsy mainly utilizes the mechanical effect of the pulsed ultrasound with lower duty cycle to further disintegrate and homogenize the target tissue.

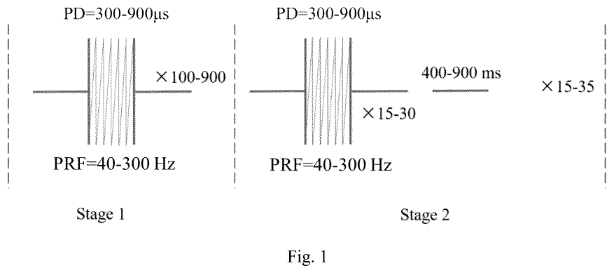

When a hundred micro-second pulsed ultrasound, whose sequence is shown in FIG. 1, is used in the two-stage pulsed ultrasound histotripsy, in the first stage, a PRF of the pulsed ultrasound is 40-300 Hz; an individual PD is 300-900 μs; and the number of repetitions S1 for each group of pulses is 100-900. In the second stage, a PRF of the pulsed ultrasound is 40-300 Hz; an individual PD is 300-900 μs; an interval after every 15-30 groups of pulses have been applied is 400-900 ms; and the number of repetitions S2 of combined pulses is 15-35.

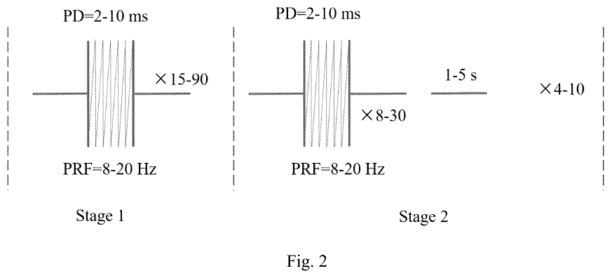

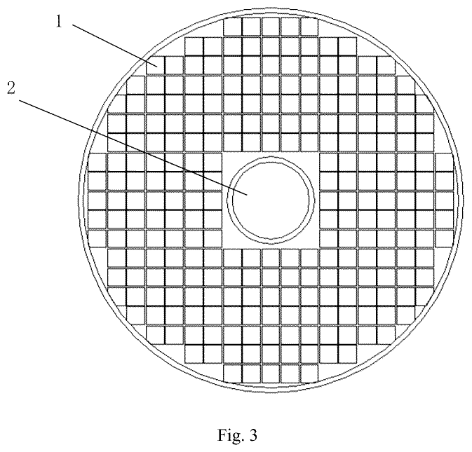

When a millisecond pulsed ultrasound, whose sequence is shown in FIG. 2, is used in the two-stage pulsed ultrasound histotripsy, in the first stage, a PRF of the pulsed ultrasound is 8-20 Hz; an individual PD is 2-10 ms; and the number of repetitions S1 for each group of pulses is 15-90. In the second stage, a PRF of the pulsed ultrasound is 8-20 Hz; an individual PD is 2-10 ms; a 1-5 s off-time is set after every 15-30 groups of pulses have been applied; and the number of repetitions S2 of combined pulses is 4-10. Unlike the hundred-microsecond pulsed ultrasound which utilizes cavitation clouds for histotripsy, the milli-second pulsed ultrasound performs efficient histotripsy mainly by enhancing the activity of boiling bubbles.

The present disclosure is described in detail hereinafter with reference to specific embodiments and the accompanying drawings.

### Example 1

A polyacrylamide gel phantom containing bovine serum albumin (BSA) is prepared. Since the BSA gel phantom has similar acoustic and thermal properties to soft tissues, it is used to simulate soft tissues. In addition, the BSA gel phantom is transparent, which makes it easy to observe the process of damage formation and bubbles. When the simulated phantom is heated above 60° C., BSA will be denatured and become opaque, which can be used as a sign of the appearance of damage, such that the damaged area can be distinguished from the normal part of the phantom.

The experimental system is built according to FIG. 9. The BSA gel phantom is adjusted to the focal point of the phased-array transducer, and the phased-array transducer is controlled to produce different focus modes as shown in FIGS. 5a-8b. Histotripsy is performed with the hundred-microsecond pulses shown in FIG. 1 and the millisecond pulses shown in FIG. 2, respectively. During the histotripsy, a high-speed camera is used to monitor in real time.

The analysis results are as follows.

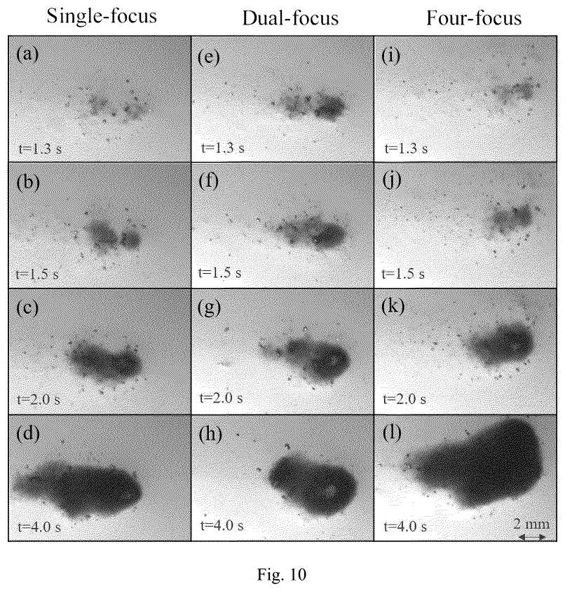

FIG. 10 shows high-speed photography results during a damage formation process using a hundred-microsecond pulsed ultrasound under single-focus mode, the dual-focus mode and the four-focus mode, where the transducer is shown at the right side. Visible damages or bubble clouds appear in the focal area around t=1.3 s under the above focus modes, and grow and expand rapidly at t=1.3-1.5 s. The left damage and the right damage begin to connect with each other around t=2.0 s, and the activity of boiling bubbles is visible within the damage. The damage expands to the surrounding area at about t=2.0-4.0 s, and forms the basic morphology at about t=4.0 s. By comparing different focus modes, it reveals that multi-focus modes can expand the damage area and effectively improve the efficiency of histotripsy.

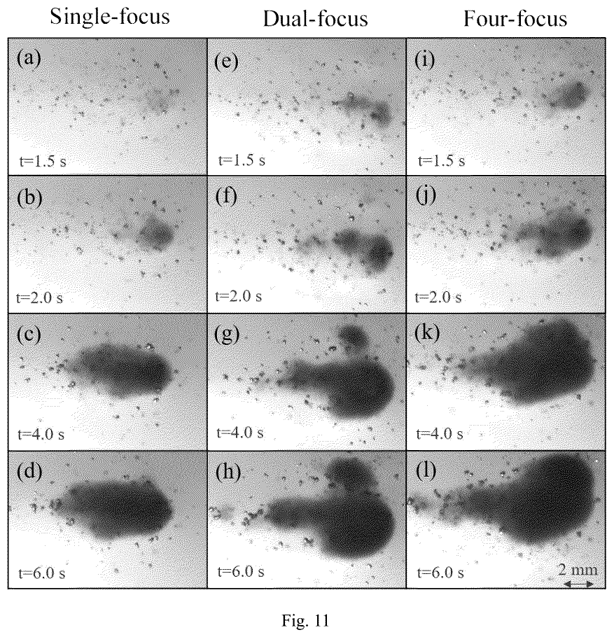

FIG. 11 shows high-speed photography results during a damage formation process using a milli-second pulsed ultrasound under the single-focus mode, the dual-focus mode and the four-focus mode, where the transducer is shown at the right side. Visible damages appear in the focal area around t=1.5 s under the above focus modes, and grow and expand rapidly at t=1.5-2.0 s. The left damage and the right damage begin to connect with each other around t=4.0 s, and the activity of boiling bubbles is visible within the damage. At the same time, under the multi-focus modes, radial damage growth is visible, and at t=4.0-6.0 s the damage expands to the surroundings, further forms the basic morphology at about t=6.0 s. By comparing the above several focus modes, it can be found that the damage volume of the multi-focus modes is larger than that of the single-focus mode in the same duration under millisecond pulses, and the damage appears to radial expansion in addition to the axial growth, and the existence of the multi-focus points can be clearly seen from the damage results. Compared to the hundred-microsecond pulse, the millisecond pulse produces a more sufficient heat, and thus the thermal damage formed in the first stage is more obvious. However, in the second stage, to avoid the occurrence of further thermal damage, the stopping time is set to be longer, and thus the overall pulse duration is also longer.

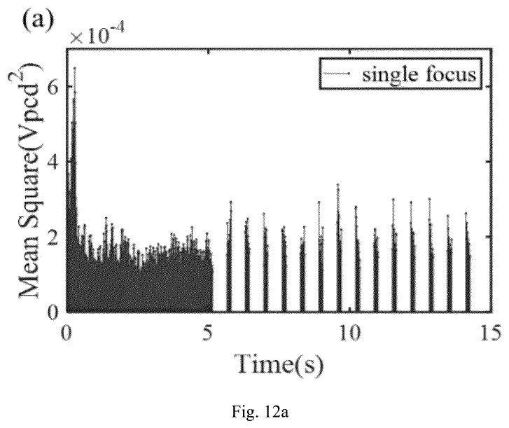

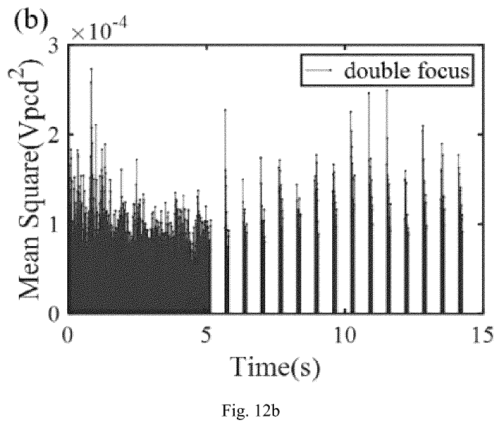

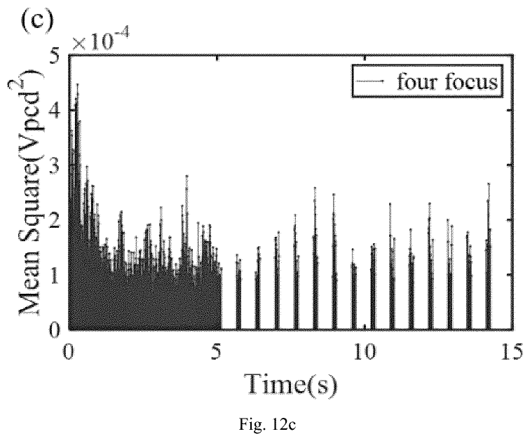

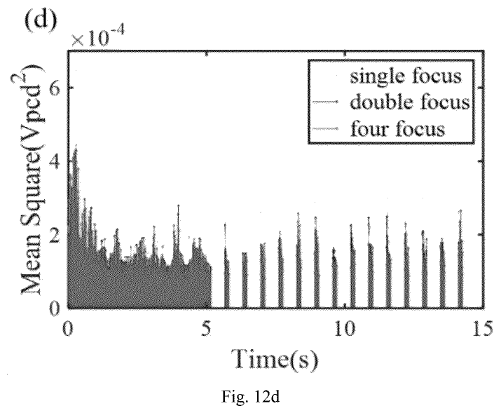

FIGS. 12a-d show changes in mean-square energy values of PCD signals with histotripsy time under the single-focus mode, the dual-focus mode and the four-focus mode. As can be seen in FIGS. 12a-d, the mean-square energy values of the above focus modes quickly reach high levels in the initial short time, which is due to the appearance of a large number of cavitation microbubbles in the focal zone at the beginning, accompanied by the process of growth, expansion, and rupture of the cavitation microbubbles. The cavitation at later times in the first stage is also maintained at a high level and there are some extremely large values, mainly due to the effect of boiling bubbles that repeatedly appear and dissipate during the damage formation process. In the second stage, no significant changes are found in the damage pattern, but the overall PCD mean-square energy remains at a high level, mainly due to the influence of mechanical effects further damaging the tissue. By comparing the above several focus modes, it shows that the cavitation effect of the single-focus mode is slightly higher than that of the multi-focus modes, which is mainly due to the fact that the acoustic field intensity of the focal zone under the single-focus mode is larger than that under the multi-focus modes under the same driving voltage, and thus the cavitation effect is more likely to occur, but the overall difference is not significant.

### Example 2

Samples with a size of 2 cm×2 cm×4 cm are cut from in vitro fresh porcine kidney and are placed in a polyacrylamide gel solution, followed by addition of a coagulant promoter to allow for gelatinization.

The experimental system is built according to FIG. 9. The in vitro porcine-kidney tissue-samples are adjusted to the focal area of the transducer, and the phased array is controlled to produce different focus modes as shown in FIGS. 5a-8b. Histotripsy is performed with the hundred-microsecond pulses shown in FIG. 1 and the millisecond pulses shown in FIG. 2, respectively.

The analysis results are as follows.

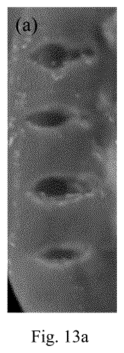

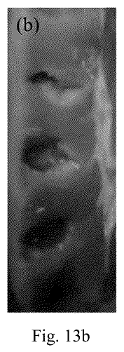

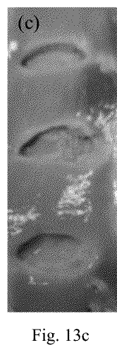

FIGS. 13a-c show histotripsy results of in vitro porcine kidney using a hundred microsecond pulses under the single-focus mode, the dual-focus mode and the four-focus mode. As can be seen from these figures, under the above focus modes, obvious cavity-like damage caused by mechanical effects are visible, and the inside of the damage is completely liquefied, but some thermal damages still remain. The multi-focus mode can expand the volume of the focal zone and improve the efficiency of histotripsy.

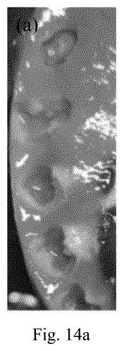

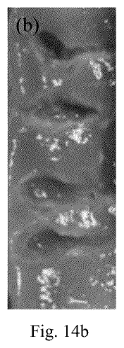

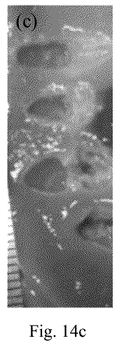

FIGS. 14a-c show histotripsy results of in vitro porcine kidney using milli-second length pulses under the single-focus mode, the dual-focus mode and the four-focus mode. As can be seen from these figures, the damage is circular under the single-focus mode, is elliptical under the dual-focus mode, and is relatively less regular under the four-focus damage, mainly due to the fact that the multi-focus modes produce a plurality of damage regions, which interact with each other, and the final damage shape is larger than that of the single-focus mode.

It should be noted that as used herein, the terms “first” and “second” are merely used for describing the purpose and distinguishing similar objects, not implying the order of precedence and not indicating relative importance. Furthermore, as used herein, unless otherwise specified, the term “a plurality of” means two or more.

It should be understood that the above description is merely illustrative, and is not intended to limit the present disclosure. It should be noted that though the present disclosure has been described in detail above with reference to the embodiments, those skilled in the art can still make various modifications, variations and replacements to the features recited in the embodiments. Those modifications, variations and replacements made without departing from the spirit of the disclosure shall fall within the scope of the disclosure defined by the appended claims.

What is claimed is:

1. A multi-focus histotripsy system based on a pulsed ultrasound phased array with hundred elements, comprising:
a transducer-wave driver subsystem;
wherein the transducer-wave driver subsystem comprises a phased-array transducer with hundred elements and a driver; the driver is connected to the phased-array transducer through an impedance matching network, wherein each of the hundred elements is connected to an independent driving channel;
the driver is a Verasonics system, and is configured to implement a focus control algorithm and set relevant parameters and ultrasound pulse modes through Matlab programming, so as to realize control of a plurality of multi-focus modes generated by the phased-array transducer and driving waveform;
the phased-array transducer is configured for histotripsy, wherein each of the hundred elements is configured to be independently controlled in terms of excitation amplitude and phase to generate acoustic distributions of the plurality of multi-focus modes, so as to form a focal region with a desired shape, size and positions for adaptively-targeted histotripsy and the phased-array transducer is configured to move a focus position electrically;
the phased-array transducer is configured to be controlled to generate the plurality of multi-focus modes through steps of:
calculating an acoustic field of the phased-array transducer; and
controlling a focus mode of the phased-array transducer using an optimization algorithm to generate the plurality of multi-focus modes;
an optimal drive signal for each of the hundred elements of the phased-array transducer under each of the plurality of multi-focus modes is configured to be calculated using a genetic algorithm focus mode to produce acoustic field distributions of a single-focus mode, a dual-focus mode and a four-focus mode;
a center of the phased-array transducer is provided with a circular hole for installing an ultrasound monitoring probe;
the multi-focus histotripsy system is configured to perform the following steps:
regulating the excitation amplitude and the phase of each of the hundred elements according to demands to control the phased-array transducer to produce the plurality of multi-focus modes; and
performing a two-stage histotripsy based on the plurality of multi-focus modes through the following steps:
in a first stage, applying a first pulsed ultrasound to an experimental sample in a target area to induce generation of boiling bubbles to preliminarily homogenize the experimental sample to form a loose product; and
in a second stage, applying a second pulsed ultrasound to mechanically disrupt and homogenize the loose product;
wherein the first pulsed ultrasound is configured to employ a first milli-second length pulse, and the second pulsed ultrasound is configured to employ a second milli-second length pulse, and the first milli-second length pulse and the second milli-second length pulse are configured to enhance activity of the boiling bubbles to perform the histotripsy; a duty cycle of the first pulsed ultrasound is 3-10%; and a duty cycle of the second pulsed ultrasound is less than 2%;
a pulse repetition frequency (PRF) of the first milli-second length pulse is 8-20 Hz; an individual pulse duration (PD) of the first milli-second length pulse is 2-10 ms; and the number of repetitions S1 for each group of the first milli-second length pulse is 15-90;
a PRF of the second milli-second length pulse is 8-20 Hz; an individual PD of the second milli-second length pulse is 2-10 ms; a 1-5 s off-time is set after every 8-30 groups of the second milli-second length pulse are applied; and the number of repetitions S2 of combined pulse is 4-10; and
the multi-focus histotripsy system is further configured to perform a step of:
before the two-stage histotripsy focus mode, adjusting a position of the experimental sample to a focal zone of the phased-array transducer through steps of:
inducing a thermal damage in the experimental sample using a continuous wave mode; intersecting two laser beams at a position suffering the thermal damage, with an intersection point serving as an approximate focal point of the phased-array transducer; and moving the experimental sample to the approximate focal point using a three-dimensional (3D) positioning subsystem.

2. The multi-focus histotripsy system of claim 1, further comprising:
a camera subsystem;
a data acquisition subsystem;
a passive cavitation detection (PCD) subsystem; and
the 3D positioning subsystem;
wherein
the PCD acoustic signal detection subsystem comprises a PCD probe, a wideband receiver, a data acquisition card and a first computer; and the PCD probe, the wideband receiver, the data acquisition card and the first computer are electrically connected in sequence; and
the 3D positioning subsystem comprises a 3D driving device and a second computer; the second computer is a control computer; the 3D driving device is electrically connected to the second computer; and the experimental sample is adapted to be set on the 3D driving device, and to be placed at a focal area of the phased-array transducer.
